(19) United States
(12) Patent Application Publication
(10) Pub. No.: US 2023/0110090 A1
(43) Pub. Date: Apr. 13, 2023

(54) System and Method of Electroporation Protocol Optimization

(71) Applicants: Life Technologies Holdings PTE Limited, Singapore (SG); Life Technologies Corporation, Carlsbad, CA (US)

(72) Inventors: Mio Xiu Lu Ling, Singapore (SG); Harmon Cosme Sicat, JR., Singapore (SG); Joshua Mead, San Diego, CA (US); Benyong Shi, Singapore (SG); Li Yong Ong, Singapore (SG); Don Paul Kovarcik, Corte Madera, CA (US); Nektaria Andronikou, Vista, CA (US)

(21) Appl. No.: 17/960,237

(22) Filed: Oct. 5, 2022

Related U.S. Application Data
(60) Provisional application No. 63/262,286, filed on Oct. 8, 2021.

(51) Int. Cl. C12M 1/42; C12M 1/36

(52) U.S. Cl. CPC C12M 35/02; C12M 1/42; C12M 41/48

(57) ABSTRACT

The present disclosure provides systems and methods of electroporation protocol optimization. Embodiments include electroporation machines capable of carrying out test protocols including multiple user-designated parameters. The protocols and parameters can be carried out on samples comprising cells, including portions of a sample, to determine optimum parameters for electroporation for different samples. The systems and methods of optimization preferably use electroporation cartridges, electroporation instruments and systems and methods of electroporation using these devices and systems. In some embodiments, electroporation cartridges comprise an electroporation chamber and electrodes.

### CROSS REFERENCE TO RELATED DATA

This application claims the benefit of U.S. Provisional Patent Application No. 63/262,286, filed Oct. 8, 2021, titled System and Method of Electroporation Protocol Optimization, the contents of which are hereby incorporated herein in its entirety.

### BACKGROUND

### Technical Field

This disclosure generally relates to systems, devices, and methods for treating cells with transient electric fields. More specifically, the present disclosure relates to systems, devices, and methods for optimizing electroporation protocols for a cell-containing fluid using electroporation systems, devices and methods.

### Related Technology

Since at least as early as the 1970's, scientists have been using electroporation as a technique for inserting molecules into animal or plant cells. By exposing cells to transient electric fields, particularly short duration, high voltage electrical fields, cellular membranes become permeable to molecules in the surrounding media, allowing cellular uptake of target macromolecules—typically proteins and nucleic acid. When the voltage and duration of exposure to electric fields is controlled appropriately, electroporated cells are able to recover membrane permeability and normal functionality. However, overexposure to electric fields—whether for extended periods of time or at too high of a voltage, can permanently disrupt the electrical potential and/or membrane integrity of the cell, leading to cell death. What is needed is a system and method for determining an optimal set of parameters, or protocol, for cellular electroporation.

### BRIEF SUMMARY

One embodiment of the present disclosure includes an electroporation system configured to test for optimal parameters for the electroporation of a sample. The electroporation system can comprise a compartment configured to removably receive an electroporation chamber, the electroporation chamber comprising a portion of the sample, the sample comprising one or more particular cell types. It can further comprise a controller communicatively coupled to the electroporation chamber, wherein the controller is configured to run one or more optimization routines, each optimization routine including a set of parameters for the electroporation of the sample to determine the optimal parameters for electroporation of the sample; wherein one optimization routing is run on the portion of the sample in the electroporation chamber.

In one aspect, the set of parameters includes voltage, pulse width, pulse number, buffer type, pulse type, and pulse interval.

In one aspect, the pulse type refers to positive or negative polarity pulse.

In one aspect, the controller is further configured to allow selection of a number of replicates for a selected optimization routine.

In one aspect, the controller is further configured to create a queue of selected optimization routines.

In one aspect, the controller is further configured to repeat individual optimization routines in the queue of selected optimization routines.

In one aspect, the controller is further configured to skip individual optimization routines in the queue of selected optimization routine.

In one aspect, the controller is further configured to re-order the selected optimization routines.

In one aspect, the controller is configured to display a step-by-step guide to a user.

In one aspect, the controller is configured to display a “quick start” option to a user, wherein the “quick start” option avoids a step-by-step guide.

In one aspect, an electroporation chamber is one electroporation chamber. In one aspect, an electroporation chamber is one or more electroporation chambers. In one aspect, one electroporation chamber is placed in a compartment at a time.

In one aspect, the electroporation chamber is within an electroporation cartridge, the electroporation cartridge comprising two electrodes.

In one aspect, the electroporation cartridge comprises at least one resealable cap.

In one aspect, at least one of the electrodes of the electroporation cartridge comprises at least part of the resealable cap.

In one aspect, the electroporation cartridge comprises a volume reducing sleeve.

In one aspect, the electroporation cartridge comprises at least one fluid overfill reservoir.

In one aspect, the electroporation cartridge comprises an authentication chip.

In one aspect, the authentication chip comprises an RFID chip.

In one aspect, the authentication chip comprises any of an erasable programmable read-only memory (EPROM), an electrically erasable programmable read-only memory (EEPROM), or flash memory.

In one aspect, the authentication chip comprises a non-floating-gate technology capable of surviving gamma radiation.

In one aspect, the electroporation chamber comprises: an elongate body; a first electrode disposed at a proximal end of the electroporation chamber; and a second electrode disposed at an opposite, distal end of the electroporation chamber.

In one aspect, the electroporation chamber comprises one or more of a non-conductive plastic, a glass, and/or a ceramic and is configured to receive a cell-containing liquid to be electroporated within the electroporation chamber.

In one aspect, at least a portion of the electroporation chamber is tapered between the first electrode and the second electrode.

In one aspect, the controller is configured to receive a command from a user to skip an optimization protocol.

In one aspect, the controller is configured to receive a command from a user to add the skipped optimization back to a queue.

In one aspect, a second optimization routine is run on a second portion of the sample in a successively received electroporation chamber.

In one aspect, the compartment removably receives one or more additional portions of the sample in the same or different electroporation chambers and wherein one or more optimization routines is run on each of the portions of samples, respectively.

Another embodiment of the present disclosure includes a method of optimizing electroporation of samples comprising cells. The method can comprise providing to a user, by an electroporation system, a list of a plurality of optimization protocols to run against one or more samples, wherein each of the plurality of optimization protocols comprises a unique combination of a plurality of parameters, wherein the electroporation system is configured to apply the plurality of parameters to the one or more samples. It can further comprise receiving, from the user, a selection of one or more of the plurality of optimization protocols; and applying, by the electroporation system in a series of operations, the one or more optimization protocols against a corresponding sample of the one or more samples such that each corresponding sample is subject to one of the one or more of the plurality of optimization protocols.

In one aspect, the plurality of parameters includes voltage, pulse width, pulse number, buffer type, pulse type, and pulse interval.

In one aspect, each of the one or more samples is comprised in an electroporation chamber.

In one aspect, each of the one or more samples comprises an authentication chip or is part of an electroporation cartridge that comprises an authentication chip.

In one aspect, the authentication chip comprises an RFID chip.

In one aspect, the authentication chip comprises any of an erasable programmable read-only memory (EPROM), an electrically erasable programmable read-only memory (EEPROM), or flash memory.

In one aspect, the authentication chip comprises a non-floating-gate technology capable of surviving gamma radiation.

In one aspect, the method further comprises receiving, by the electroporation system, a number of replicates to run and applying, for each optimization protocol, the number of replicates against one of the one or more samples.

In one aspect, the method further comprises displaying, to a user, instructions on opening the system, inserting an electroporation cartridge into the system, closing the system and running the optimization protocols.

In one aspect, the method further comprises creating a queue of selected optimization routines.

In one aspect, the method further comprises receiving a request, from a user, to repeat a selected optimization routine in the queue.

In one aspect, the method further comprises receiving a request, from a user, to skip a selected optimization routine in the queue.

In one aspect, the method further comprises receiving a request, from a user, to re-order the selected optimization routines in the queue.

In one aspect, the method further comprises displaying, to a user, a status of each selected optimization routine in the queue.

In one aspect, status can be at least one of; completed, skipped, and in progress.

In one aspect, the one or more samples comprise one or more portions of a sample.

Another embodiment of the present disclosure includes a method for optimizing electroporation of a sample comprising cells. The method can comprise receiving, by an electroporation system, a choice of one or more optimization protocols to run against one or more samples, wherein each of the one or more optimization protocols comprises at least a voltage, a pulse width, and a number of pulses. It can further comprise receiving, by the electroporation system, a number of replicates to run, wherein the number of replicates multiplied by the one or more optimization protocols equals a number of trials. It can further comprise receiving, by the electroporation system in a series of electroporation operations corresponding to a number of selected optimization protocols and the number of replicates, an electroporation chamber containing one of the one or more samples for each of the number of trials; and applying to each respective sample, by the electroporation system, the respective optimization protocol.

In one aspect, each of the one or more samples is received in an electroporation cartridge.

In one aspect, the method further comprises sending, by the electroporation system, results of each of the one or more samples to a remote machine for analysis.

In one aspect, each of the one or more samples is contained in an electroporation chamber of an electroporation cartridge.

In one aspect, each of the one or more samples comprises a cell population having a particular cell type.

In one aspect, each of the one or more samples comprises a cell population having a plurality of cell types.

In one aspect, the method further comprises assessing, after each trial, a level of electroporation in cells of the respective one or more samples; and assessing, after all trials have completed, which optimization protocol achieved the best electroporation.

In one aspect, the method further comprises assessing, after each trial, one or more of electroporation efficiency, cell viability, and expression of electroporated cells.

In one aspect, the electroporation system is configured to assess risk of arcing for each optimization protocol.

In one aspect, the method further comprises recording, by the electroporation system, information from an authentication tag associated with each of the one or more samples.

In one aspect, the one or more samples comprise one or more portions of a sample.

Accordingly, systems, methods, and devices for automated electroporation of cell-containing fluid are disclosed.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

The foregoing has outlined rather broadly the features and technical advantages of the present invention in order that the detailed description of the invention that follows may be better understood. Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only exemplary embodiments of the disclosure and are not therefore to be considered to be limiting of its scope. The disclosure will be described and explained with additional specificity and detail through the use of the accompanying drawings in which:

FIG. 1 illustrates an embodiment of a user interface for an electroporation system;

FIGS. 2A-2E show several views of an exemplary electroporation cartridge having an electroporation chamber, in accordance with some embodiments of the present disclosure;

FIG. 3 shows an embodiment of an electroporation system having a lid in an open position;

FIG. 4 illustrates a method embodiment of optimizing electroporation protocols under the current disclosure;

FIG. 5 illustrates a method embodiment of optimizing electroporation protocols under the current disclosure;

FIG. 6 is a cross-section of another exemplary electroporation cartridge having an authentication chip associated with the electrode cap in accordance with some embodiments of the present disclosure;

FIGS. 7A-7D show several perspective views of another exemplary electroporation cartridge having an authentication chip associated with the electrode cap and a gripping member associated with the electroporation chamber body in accordance with some embodiments of the present disclosure;

FIG. 8 illustrates a schematic of an electroporation circuit, according to one embodiment;

FIG. 9 illustrates an exemplary method flow for electroporation of cells prepared in accordance with one or more embodiments of the present disclosure;

FIG. 10 illustrates a flow chart for a user interface embodiment for an electroporation system under the present disclosure;

FIG. 11 illustrates a flow chart for a user interface embodiment for an electroporation system under the present disclosure; and

FIG. 12 illustrates a flow-chart for a method embodiment under the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Embodiments under the present disclosure include protocol optimization systems and methods for measuring, optimizing and testing the levels of electroporation in cell cultures. Electroporation is a process in which an electrical field can be applied to cells so as to increase the permeability of the cell membrane. Increasing the permeability can be useful for introducing drugs, DNA, proteins, peptides, and other biomolecules, or other chemicals or substances into the cell. The level of electroporation in a cell culture can be given by several different metrics, including: efficiency of electroporation, cell viability after electroporation, expression of gene or protein of interest in electroporated cell, knockout of a gene of interest, or change in genotype or phenotype of a cell. A given user may use different of these metrics depending on their specific needs. Other metrics or combinations of the preceding, are possible as well. Achieving efficient electroporation with good cell viability can be difficult so it is valuable to understand what parameters impact the electroporation process. Important parameters include voltage, electrical pulse width, electrical pulse number, buffer type, electrical pulse type, and time interval between pulses. Systems and methods described herein include the ability to select from sets of preprogrammed parameters, or to choose parameters, to apply across a number of cell culture samples. Those samples can then be tested to determine which parameters, or protocols, produced the most effective results. This can assist users in better understanding, for a given type of cell, cell population, or cell culture, what parameters lead to the best electroporation. Different cells, cell populations, or cell cultures may be from individuals of unique DNA, from human subjects, from different animals, different bacteria, or other types of cells for which electroporation testing is desired.

The methods and systems described herein can be used for electroporation in various types of cells. Cells tested or otherwise analyzed can include a single type of a cell, a cell population, cells grown in a culture, a mixture of different cells, bone marrow cells, blood cells, stem cells, or any specific type of cell that a user wishes to analyze.

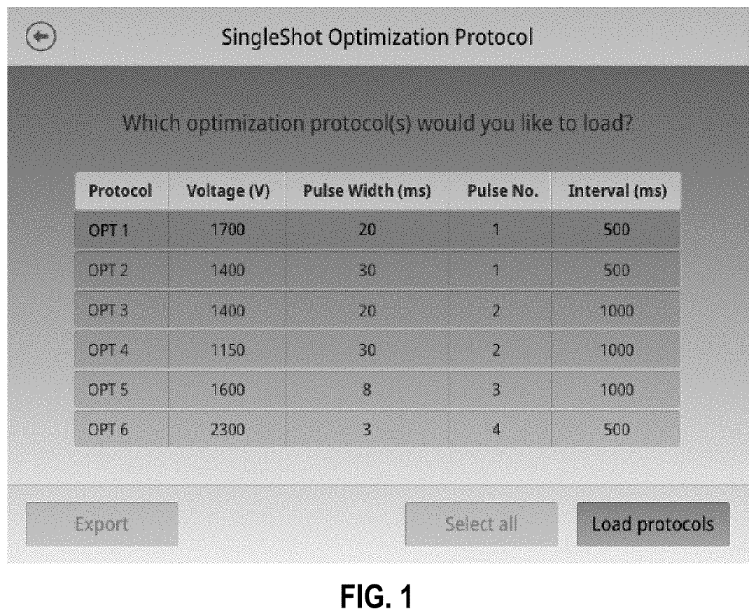

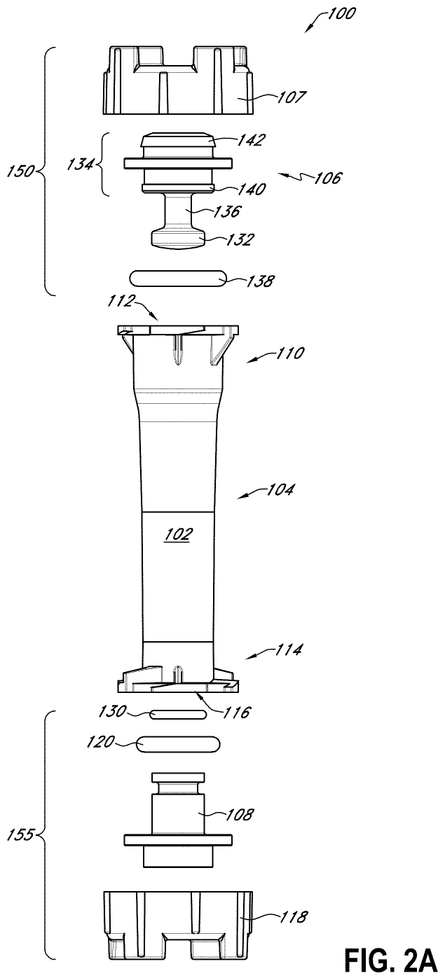

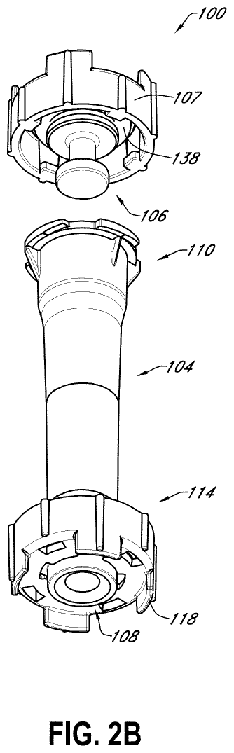

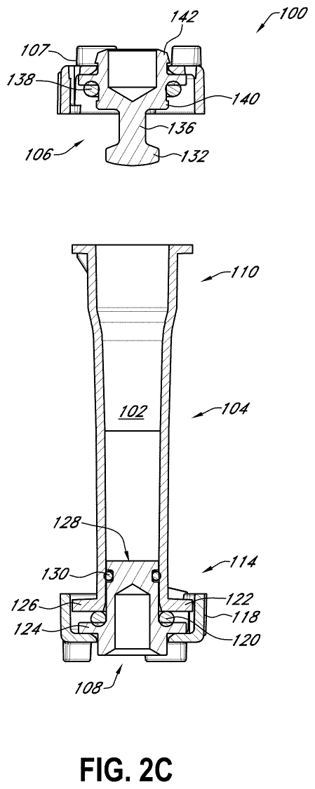

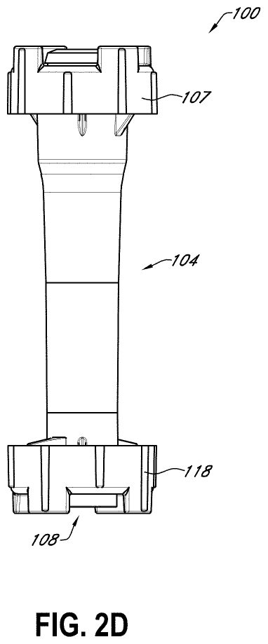

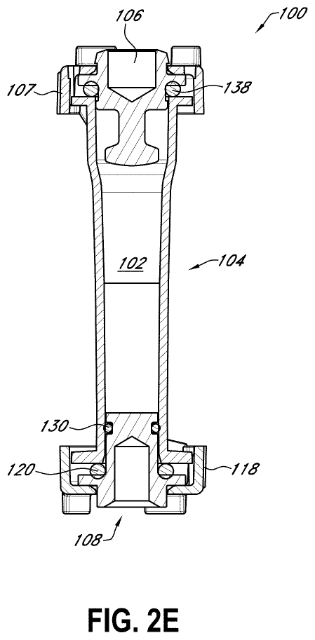

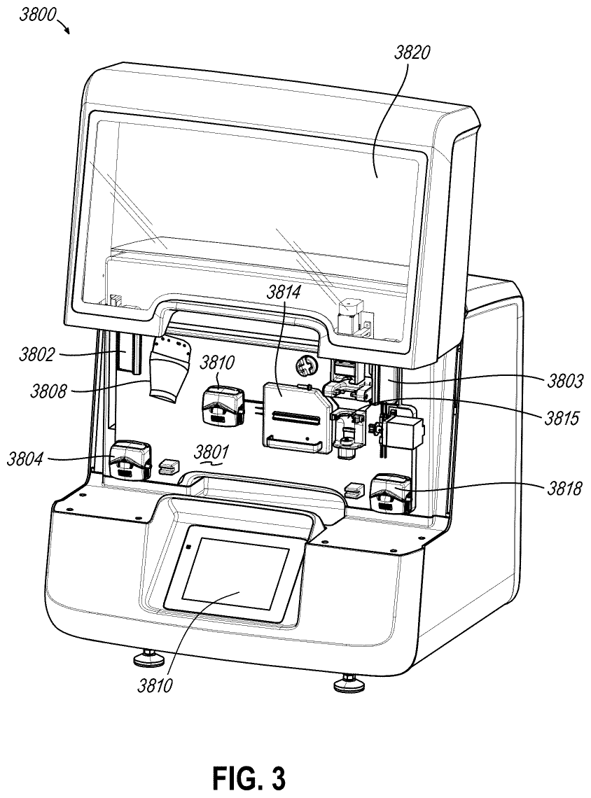

For example, a user might wish to test six different protocols, each protocol comprising a unique set of parameters. FIG. 1 shows a set of possible protocols OPT 1-OPT 6. Voltage, pulse width, pulse number, and interval are varied as shown. A possible electroporation cartridge 100, containing a sample or a portion or an aliquot of a sample comprising cells to be electroporated, is shown in FIGS. 2A-2E and will be described further below. FIG. 3 shows a possible electroporation system 3800 that can receive an electroporation cartridge 100 with cells inside of it. Electroporation system 3800 can apply each of OPT 1-OPT 6 to a different sample or to a portion of a sample in an electroporation chamber contained in the electroporation cartridge 100 and measure resulting levels of electroporation or other variables. Another electroporation cartridge 100 with another sample or sample portion can be inserted to the machine by a user who inserts a new electroporation cartridge 100 for each trial or operation. Each new trial or operation can test a new set of parameters on a different cell sample in a unique electroporation cartridge 100.

FIG. 3 illustrates one example embodiment of an electroporation system 3800 that includes a modular casing 3803 and an instrument panel 3801. Electroporation system 3800 can comprise an instrument panel 3801 configured to receive an electroporation chamber comprising a sample of cells to be electroporated. In some embodiments, an electroporation compartment 3815 is configured to receive one or more types of electroporation cartridge having an electroporation chamber configured for holding a sample for electroporation. Electroporation system 3800 can further comprise tubing having an inlet end and an outlet end, the tubing being routed through the casing to fluidically connect the plurality of electroporation system components and flow sensors configured to detect flow through particular section of the tubing. Electroporation system can also comprise a controller communicatively coupled to the one or more pumps and the flow sensors, wherein the controller is configured to run two or more optimization routines on a series of samples of one or more particular cell types, each optimization routine including a set of parameters for the electroporation of one of the set of samples such that each sample of the set of samples may be tested to determine the optimal parameters for a particular cell type. User interface/controller 3810 of electroporation system 3800 can allow users to select protocols, choose replicate numbers for each protocol, or adjust other parameters of electroporation system 3800. For example, user interface 3810 can display some of the methods and user interfaces described below.

Application of voltage to a cell culture can be achieved via the electrodes 106, 108 of electroporation cartridge 100 when inserted or combined with electroporation system, such as in FIG. 3. One trial or operation will preferably comprise the application of one optimization protocol and its comprising parameters or variables. After each operation, a user may replace the electroporation cartridge for the running of a new optimization protocol, or a replicate of a previous optimization protocol.

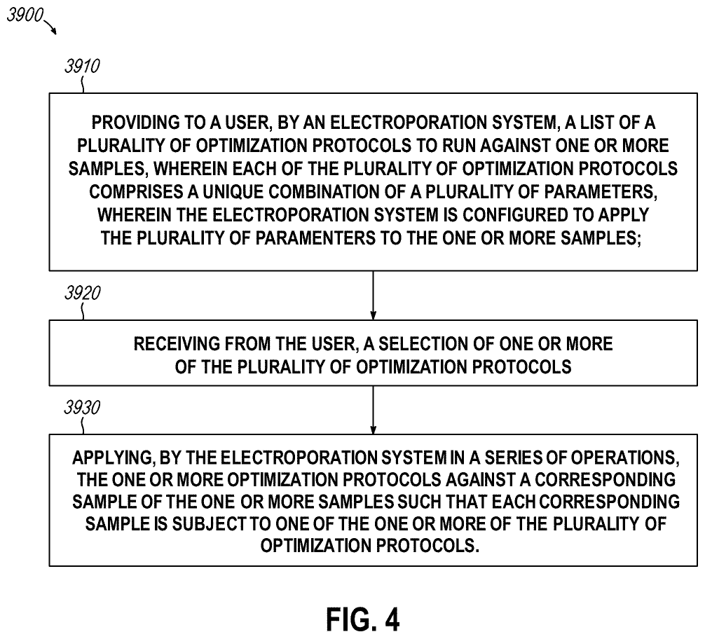

Another possible embodiment under the present disclosure comprises a method of optimizing electroporation of samples comprising cells 3900, shown in FIG. 4. Step 3910 is providing to a user, by an electroporation system, a list of a plurality of optimization protocols to run against one or more samples, wherein each of the plurality of optimization protocols comprises a unique combination of a plurality of parameters, wherein the electroporation system is configured to apply the plurality of parameters to the one or more samples. Step 3920 is receiving, from the user, a selection of one or more of the plurality of optimization protocols. Step 3930 applying, by the electroporation system in a series of operations, the one or more optimization protocols against a corresponding sample of the one or more samples such that each corresponding sample is subject to one of the one or more of the plurality of optimization protocols.

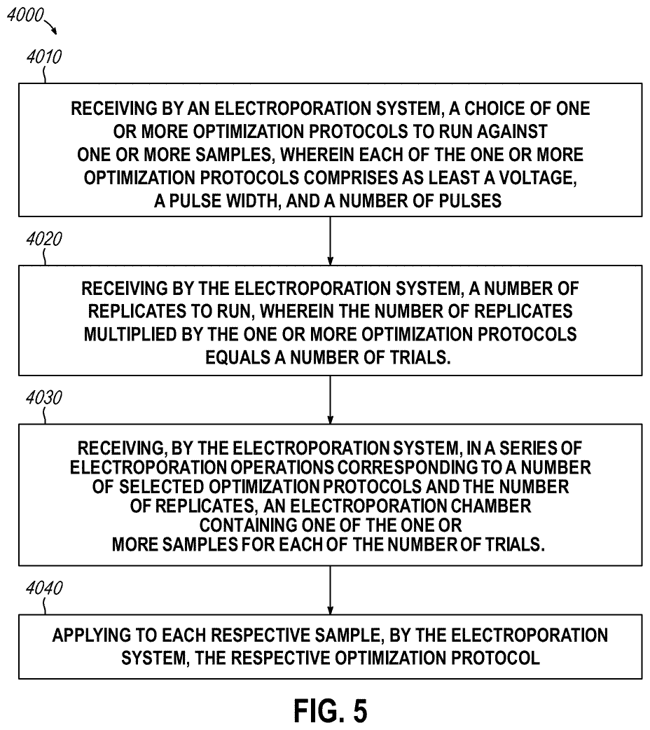

Another possible embodiment comprises a method for optimizing electroporation of a sample comprising cells 4000, shown in FIG. 5. Step 4010 is receiving, by an electroporation system, a choice of one or more optimization protocols to run against one or more samples, wherein each of the one or more optimization protocols comprises at least a voltage, a pulse width, and a number of pulses. Step 4020 is receiving, by the electroporation system, a number of replicates to run, wherein the number of replicates multiplied by the one or more optimization protocols equals a number of trials. Step 4030 is receiving, by the electroporation system, in a series of electroporation operations corresponding to a number of selected optimization protocols and the number of replicates, an electroporation chamber containing one of the one or more samples for each of the number of trials. Step 4040 is applying to each respective sample, by the electroporation system, the respective optimization protocol.

Embodiments of the present disclosure include electroporation chambers, electroporation cartridges, associated systems, and methods for using the same that enable a reduction of bubble formation during electroporation, even at large volumes that would traditionally cause arcing (e.g., 1 mL or greater). In addition, embodiments described include methods of protocol optimization for the use of electroporation systems and cartridges, to determine those parameters that are most conducive to electroporation for any particular type of cell.

An exemplary electroporation cartridge is illustrated in FIGS. 2A-2E, where FIG. 2A provides an exploded view of the components of the exemplary electroporation cartridge, FIGS. 2B and 2C show the electroporation cartridge of FIG. 2A in a partially assembled and an uncapped position, and FIGS. 2D and 2E show surface shaded and cross-sectional views of the assembled cartridge in a capped position. The illustrated electroporation cartridge, and other electroporation cartridges described herein, in some embodiments, may include one or more security features for ensuring that they are only used a single time. For example, a single use electroporation cartridge may include a locking feature, breakaway feature, and/or other mechanical feature that does not allow reuse of the electroporation cartridge after it has been inserted and removed from the electroporation system. Single use of the single use electroporation cartridges may additionally or alternatively be ensured electronically, such as through an electronic tag or code associated with each single use electroporation cartridge and scanned/logged by the electroporation system.

According to one embodiment, as shown in FIGS. 2A-2E, an exemplary electroporation cartridge 100 includes an electroporation chamber 102 defined by an elongate body 104, a first electrode 106 disposed at a proximal end 110 of the electroporation chamber 102, and a second electrode 108 disposed at an opposite, distal end 114 of the electroporation chamber 102, wherein at least one of the first electrode 106 or second electrode 108 is moveable between a capped position for electroporation and an uncapped position for loading a sample (e.g., as shown between FIGS. 2B-2E). As particularly shown in FIG. 2A, the elongate body has two open ends— a proximal end 110 defining a proximal opening 112 and a distal end 114 defining a distal opening 116—where first electrode 106 and second electrode 108 are installed, respectively. According to one embodiment, first electrode 106 is associated with a removable cap 107 that allows for selective association of first electrode 106 with elongate body 104. In some embodiments, second electrode 108 of cartridge 100 is inserted into the distal opening 116 of elongate body 104 where it is secured in place by distal cap 118. In some embodiments, second electrode 108 is secured within and/or in association with distal end 114 of elongate body 104 by distal cap 118 via a locking feature to prevent the lower cap from being removed. This can advantageously act to reduce potential confusion about which end a user is to be using for adding and/or removing cell containing fluid for electroporation and also provides the benefit of a modular construction that can be partially assembled to create a more structurally secure cartridge.

As shown in FIG. 2C, second electrode 108 additionally comprises a first sealing member 120 disposed between the second electrode 108 and a distal surface 122 of elongate body 104, the first sealing member operable to form a fluid tight connection between second electrode 108 and distal surface 122 of elongate body 104. In some instances, the fluid tight connection is provided by compressing a sealing member (e.g., an O-ring or other gasket) between an electrode flange 124 and a distal body flange 126 oriented in a plane substantially parallel to the electrode flange 124. In some embodiments, distal cap 118 locks into association with the elongate body with the sealing member 120 disposed between the electrode flange 124 and the distal body flange 126, forming the fluid tight connection therebetween.

With continued reference to FIG. 2C, in some embodiments, second electrode 108 can include a protruding portion 128 that extends into electroporation chamber 102 from distal end 114 of elongate body 104 to define the bottom of electroporation chamber 102. In some embodiments, second electrode 108 can additionally include a second sealing member 130 disposed about the protruding portion 128 and positioned distal to the proximal surface of the second electrode where it forms a fluid tight connection with the interior sidewall of the electroporation chamber 102. Whereas the first sealing member 120 forms a fluid tight seal between second electrode 108 and elongate body 104 via compression of distal cap 118 to seal the distal end of the electroporation chamber 102 from the outside environment, the second sealing member 130 forms a fluid tight connection between the protruding portion 128 of second electrode 108 and the interior sidewall of the electroporation chamber 102 to minimize dead volume by creating a seal closer to the wetted proximal surface of the second electrode 108 to prevent fluid from seeping around the second electrode 108.

In some embodiments, such as that illustrated in FIG. 2C, a circumference of the protruding portion 128 can have a shape complementary to the contour of the inner surface of the elongate body 104 that defines electroporation chamber 102 such that a diameter of the proximal end of second electrode 108 is substantially equal to a cross section of electroporation chamber 102. Additionally, the proximal surface of the second electrode can be a flat, uniform surface positioned orthogonal to a longitudinal axis of the electroporation chamber.

By including one or more of the foregoing structural features in the shape and/or location of the second electrode with respect to the electroporation chamber, certain benefits can be derived. For example, generation of a uniform electric field is one factor for successful and efficient electroporation. To have a uniform electric field, it is advantageous for the opposing first and second electrodes to be substantially parallel and having essentially the same cross-sectional geometry as the electroporation chamber. A uniform electric field is most effectively generated within an electroporation chamber having a uniform cross section (e.g., a constant diameter). Accordingly, in some embodiments, the electroporation cartridges disclosed herein can include an electroporation chamber having a uniform cross section along a length of the reaction chamber. The uniform cross section may extend an entire length of the electroporation chamber between the first and second electrodes such that the electroporation cartridge is configured to produce a uniform electric field within the electroporation chamber disposed between the first and second electrodes. As a non-limiting example of the foregoing, the electroporation chamber may be defined as a cylindrical cavity having a circular cross section extending along the entire length of the cylindrical cavity between the first and second electrodes.

Alternatively, the uniform cross section may extend along a length less than the entire length of the electroporation chamber. In such embodiments, at least a portion of the electroporation chamber can be tapered between the first electrode and the second electrodes. Preferably, the tapered portion of the electroporation chamber does not substantially interfere with generation of a uniform electric field between the first and second electrodes. For the purposes of this disclosure, the tapered portion of the electroporation chamber does not substantially interfere with generation of a uniform electric field if the electric field generated between the opposing first and second electrodes is defined by field lines that are substantially parallel and equally spaced within a 10% degree of tolerance. For clarity, a taper within the electroporation chamber can include a narrowing of a proximal sidewall defined between the proximal opening of the elongate body and an inflection point on the sidewall defining the electroporation chamber such that the proximal sidewall narrows from a first diameter defined by the proximal opening to a second, smaller diameter defined at a position distal to the inflection point.

It should be appreciated that the presence of a taper or uniform cross section along an entire length of the electroporation chamber can affect the methods available to manufacture the electroporation chamber efficiently and/or cost-effectively. Preferably, the elongate body and/or electroporation chamber is made of or includes a non-conductive irradiation-stable plastic, ceramic, and/or glass. For example, the elongate body and/or electroporation chamber can be made of polycarbonate or another non-conductive gamma-stable plastic. Alternatively, glass and ceramic are both electrically insulative and thermally more conductive than polycarbonate; advantageously, both materials can be mass produced with zero draft sidewalls and provide a constant cross section.

In some embodiments, the chamber is made of material that can be sterilized by one or more of steam sterilization, flash sterilization, hydrogen peroxide sterilization, vaporized hydrogen peroxide sterilization, gamma ray sterilization, peracetic acid sterilization, ethylene oxide sterilization, chlorine dioxide gas sterilization, electron beam sterilization, or the like, without compromising the functionality of the chamber (e.g., without reduction in electroporation efficiency or impacting cell viability).

As provided by the foregoing, a uniform electric field can be generated between two opposing electrodes having cross sections that are close to the shape and size of the uniform cross section of the electroporation chamber. The inventors found, however, that when making various cartridges having these features and geometry, air tended to be trapped more easily when sealing the chamber. Trapped air can cause the breakdown of electrical conductivity within the electroporation chamber and result in arcing, which negatively affects the electroporation process and performance.

To overcome the air trap problem, the electroporation cartridges disclosed herein make use of surface tension at liquid to air interfaces where there is greater attraction of liquid molecules to each other than to the molecules in the air. This results in formation of a convex shaped meniscus, and upon capping the top electrode, the liquid is displaced around the electrode so that no air is trapped between the sample and the distal surface of the first electrode. To encourage this, the distal portion of first electrode 106 can have a bell shape or bulbous protrusion 132 separated from a base region 134 by a narrow stem 136, as shown in FIGS. 2A-2E. The bulbous protrusion 132 can have a smaller diameter than the cross section of the electroporation chamber 102 so as to form a gap therebetween. This gap between the bulbous protrusion and the chamber body allows small bubbles to exit the electroporation volume. The narrow stem 136 can allow for a greater volume of air in the proximal portion of the chamber, which can serve as a compressible volume during electroporation to minimize the pressure buildup within the chamber caused by the partial vaporization of the sample.

The bell or bulbous shape of the distal end of the first electrode can provide additional advantages during electroporation. Bubbles can form during electroporation by electrolysis and/or vaporization of water. The bulbous extension can be operable to displace one or more bubbles generated during electroporation so that they are removed from the electrode surface before agglomerating into a bubble of sufficient size to cause arcing. For example, the bulbous extension may have an arcuate or convex surface that encourages any bubbles rising from the electroporation volume to pass along the surface of bulbous extension and rise to the sample-air interface proximate the stem.

Further, it was beneficially found that under higher pressures, the intensity of vaporization could be decreased, leading to smaller and/or less bubble formation during electroporation. By making the electroporation chamber sealed prior to electroporation, the release of energy and electrolysis occurring during electroporation will make the chamber behave like a pressure chamber. Any increase in pressure within the chamber will delay the formation of bubbles reaching a significant size that could result in arcing.

Accordingly, in some embodiments, such as that shown in FIG. 2A-2E, the first electrode 106 is associated with a sealing member 138 operable to form a fluid tight connection between the first electrode and the elongate body 104. In some embodiments, the axial compression for maintaining the fluid tight seal can be provided by the removable cap 107 associated with the first electrode 106. In some embodiments, the removable cap 107 is threadedly sealed to the elongate body 104, though it should be appreciated that other forms of connection are contemplated herein (e.g., friction fitting, snap fitting, etc.). As shown in FIG. 2A-2E, each of the caps 107, 118 feature flanges that extend beyond the outer surfaces of the electrodes. These flanges can provide the additional balance and stability to the device, such as when placed on a flat surface. Absent the illustrated flanges, the narrow footprint and the midline center of gravity of the cartridge is likely to make the cartridge unstable.

Because the first electrode 106 is removed by the user during normal operation, there may be a tendency for the sealing member to be lost or disassociated with the electrode when uncoupled from the elongate body. To prevent this, the electrode may include a retaining feature 140 proximal to the stem 136 that is configured to allow the sealing member 138 to stretch over the retaining feature 140 but then be retained thereby adjacent to the sealing surface of the first electrode 106. This sealing surface creates a functionally closed system at the upper end of the chamber when the cap 107 is associated therewith.

In some embodiments, the first electrode 106 can additionally include a cap retaining feature 142 to prevent the end cap from separating from the electrode once installed. The cap retaining feature 142 may act as a barb for a snap fit, may threadedly secure to the cap, or may be retained thereby by any other means known in the art. Notably, in some embodiments, it may be beneficial to secure the first electrode to the cap in such a way that the cap rotate independently from the first electrode so that the associated sealing member only receives axial compression.

In some embodiments, the electrodes 106, 108 are made from, or plated with, a conductive material that does not negatively impact cells by introducing harmful or toxic elements either passively or during electroporation. For example, plating the electrode in pure gold can provide an electrode with beneficial conductive properties that is unlikely to introduce harmful or toxic elements into the electroporation media. Further, it should be appreciated that the electrodes 106, 108 can be connected to a high voltage circuit and either may act as the anode or cathode or may alternate between the two, depending on the electroporation protocol. Alternatively, other non-toxic and/or non-reactive metals or materials can be used, as known in the art.

In some embodiments, the volume within an electroporation chamber of the present disclosure is larger when compared to prior art electroporation cuvettes. In some embodiments, electroporation chambers, have an internal volume from about 10 mL to about 1 mL. In some embodiments, electroporation chambers, have an internal volume from about 1 mL to about 100 μl.

In some embodiments, exemplary electroporation chambers of the present disclosure can have a volume less than about 5 mL, preferably less than about 3 mL, or in some embodiments, less than about 4 mL, less than about 2 mL, or less than about 1 mL. In some embodiments, where the internal volume of an electroporation chamber is from about 10 mL to about 1 mL, at these volumes and at the preferred range of voltage for electroporating most cellular samples, the distance between the first and second electrodes is between about 20 mm-100 mm, e.g., between about 30 mm-50 mm, between about 40 mm-70 mm, and/or between about 60 mm and about 100 mm.

In another exemplary embodiment, the volume of the electroporation chamber is about 1 mL or between about 100 μL-1 mL. In some embodiments, at this volume and at the preferred range of voltage for electroporating most cellular samples, the distance between the first and second electrodes is between about 20 mm-40 mm, e.g., between about 22 mm-38 mm, between about 25 mm-35 mm, and preferably about 30 mm. Having an electroporation chamber configured to this size is advantageous because a standard 1 mL pipette can easily enter the body of the 1 mL electroporation chamber and thus aspirate nearly 100% of the electroporated sample and no specialized equipment is necessary beyond that which is commonly used for moving such volumes of fluid about in a laboratory or clinical setting.

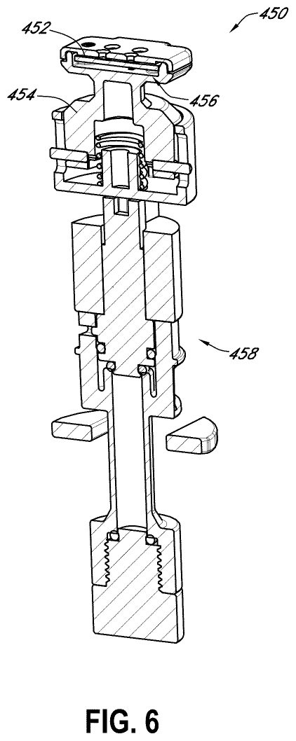

Referring now to FIG. 6, illustrated is a cross-section of an exemplary electroporation cartridge 450. The components and functionality of cartridge 450 may be similar to those described above, with the addition of an authentication chip 452 in association with an electrode cap 454. The authentication chip 452 can be any form of authentication or use-limiting device known in the art and can impart any of a number of desired functionalities to the disclosed cartridges and systems. For example, the authentication chip can be, or include, non-volatile memory, which can be used to embed manufacturing characteristics and operating parameters, data storage, security, or to manage limited use or reuse of the associated cartridge. This can beneficially protect against use of unauthorized aftermarket consumables and/or ensure authenticity and use of cartridges made by the original equipment manufacturer. Non-volatile memory can also provide the added functionality of allowing factory calibration of the cartridge such that it can communicate a given run protocol to the electroporation system to which it is associated during use. In this way, the cartridge may specify one or more runtime parameters of the electroporation system and/or can be optimized for use with various cell type(s) or electroporation target(s).

Authentication chips can take a variety of forms and may, in some instances, depend on the type of sterilization protocol (if any) used in manufacturing and/or packaging the cartridges. In general, gamma radiation—a common sterilizing agent in manufacturing of medical or laboratory grade equipment—is directly incompatible with semiconductor devices that traditionally incorporate floating-gate memory technologies used in many nonvolatile memories, such as erasable programmable read-only memory (EPROM), electrically erasable programmable read-only memory (EEPROM), and flash memory. In embodiments where sterilization via gamma radiation is desired, alternative non-floating-gate technologies can be used, including those user-programmable nonvolatile memory devices known in the art.

Other forms of authentication chips can be used and are within the scope of this disclosure. For example, simple electronics can be used as a reliable method for limiting cartridges to a single use or a prescribed number of uses. In an exemplary use, a cartridge utilizing a fuse-incorporated circuit within the authentication chip can be associated with an electroporation system. After running a given electroporation protocol with the associated cartridge, the electroporation system can send a high current through the authentication chip to blow the fuse. If the cartridge is removed and later reconnected, the lack of electrical continuity through the fuse can be a signal to the associated electroporation system to remain inactive, can prevent the system from completing a requisite circuit for functionality, or the like.

Alternative authentication chips also include radiofrequency identification (RFID) tags. An RFID tag can be associated with each cartridge and when the cartridge is loaded into a compatible electroporation system that has an RFID reader, the RFID tag can communicate information to the electroporation system that is relevant to the given cartridge. In this way, RFID tags can beneficially provide both use-limiting and counterfeit prevention capabilities. For example, the RFID tag can include the type of cartridge associated therewith, the number of times the cartridge can/has been used, a key or manufacturer specific authentication, or the like.

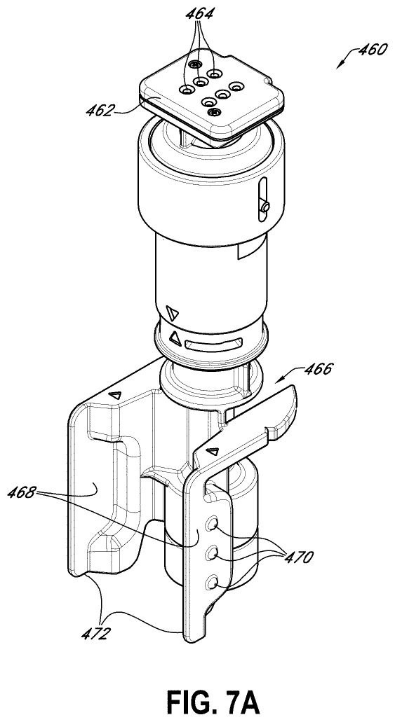

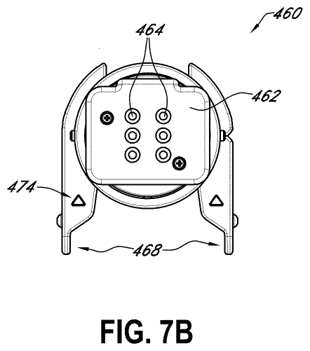

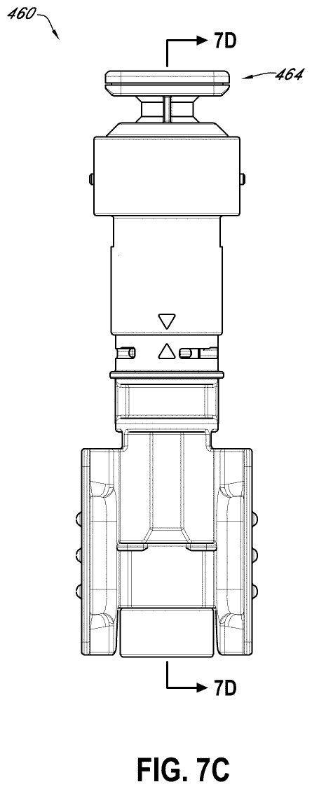

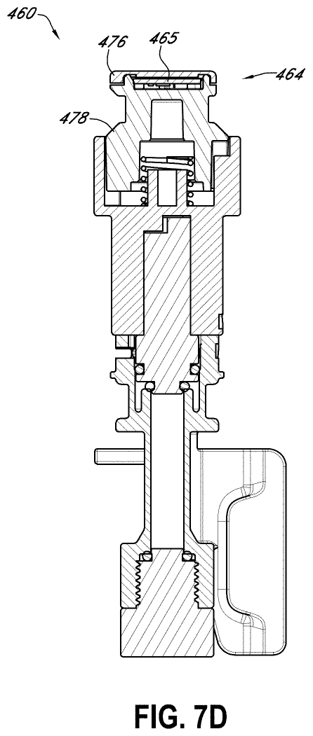

With continued reference to FIG. 6, authentication chip 452 can be housed within a space 456 formed by electrode cap 454. Access to the chip (e.g., for communicating with the electroporation system) can be achieved through contactless communication protocols or through one or more pin ports formed within electrode cap 454. FIG. 7A-7D illustrate various views of another exemplary single use electroporation cartridge 460 having an authentication chip 465 housed within electrode cap 462. As illustrated in the cross-section of FIG. 7D, authentication chip 465 can rest within a recess formed by a lower portion 478 of electrode cap 462 and is retained therein by an upper portion 476 of electrode cap 462. The upper portion 476 can, in some embodiments, be secured to the lower portion 478 in a manner that prevents tampering or otherwise removing authentication chip 465 from electrode cap 462 following manufacturing.

The following provides an initial overview of an exemplary electroporation system and corresponding process for electroporating a sample. As will be appreciated, embodiments described herein provide for effective electroporation of relatively high volumes of sample, and are capable of providing high electroporation efficiency, high cell viability, and a safe and relatively straightforward user experience, among other benefits. As will be seen in the more detailed description below, systems may be functionally closed such that all contact parts are closed off from the ambient environment, thus limiting potential contamination and also enhancing safe operation of the device. Further, as detailed below, disclosed systems are capable of recovering sample in the case of a system error, overly high temperature reading, arc risk reading, and/or other warning event. In the case of such an event, for example, the relevant sub-volume(s) of the sample may be pumped out of an electroporation chamber and back into a mixing reservoir, or even back into the sample input bag from which it originated.

Arcing negatively affects both cell viability and transfection efficiency. Often, samples intended for electroporation are valuable, so it is desirable to keep waste and/or yield loss to a minimum. The major cause of arcing is bubble formation. The systems and methods of the present invention beneficially include features that reduce the occurrence of arcing or that at least detect the risk of arcing and allow sample recovery prior to wasting a portion of sample as a result of arcing. Under high voltage applications like electroporation, nearly any bubble of significant size will result in arcing. It is known that bubbles form as a result of heat generated from electrical discharge through an aqueous cell containing solution. This intense, often repeated heating can also cause bubble formation through localized vaporization of water molecules. The electrical discharge through the cell containing solution that occurs during electroporation can cause electrolysis of water molecules, forming oxygen and hydrogen gas—another source of bubbles during electroporation.

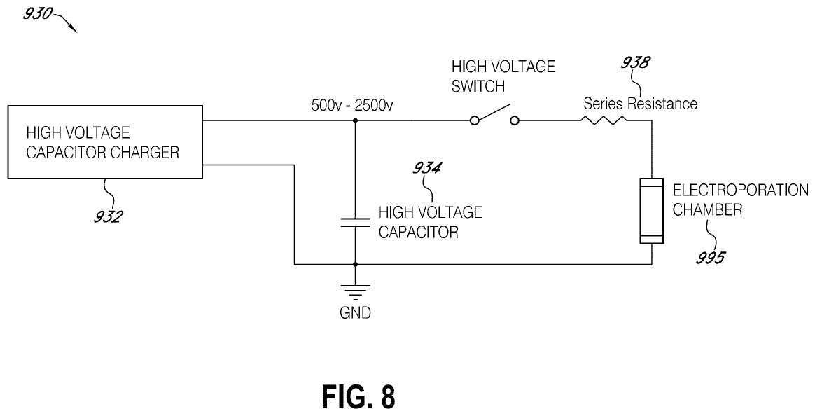

FIG. 8 illustrates a schematic of an electroporation circuit 930 comprising a charger 932 and capacitor 934 electrically connected to the electroporation chamber 995. A high voltage electroporation system may utilize a voltage pulse in the range of about 500 V to about 2,500 V. achieving electrical pulses based on such high voltages with repeatability and accuracy is desirable. Calibration of the charger 932 and/or capacitor 934 may reduce some variability. However, as indicated in the schematic circuit 930, there will be an inherent amount of inherent circuit resistance 938. This may be due to circuit protection components, discharge resistors or other safety features of the circuit, and/or the resistance of the high voltage switch when in the on position, for example. In addition, the resistance of the electroporation chamber 995 will vary according to the sub-volume properties and temperature. For example, for a chamber volume of 1 mL, the resistance may commonly vary from about 500 Ohm to 2,000 Ohm. The variability in resistance from one sub-volume to another can lead to inconsistent electrical pulses, which in turn can lead to inconsistent electroporation results.

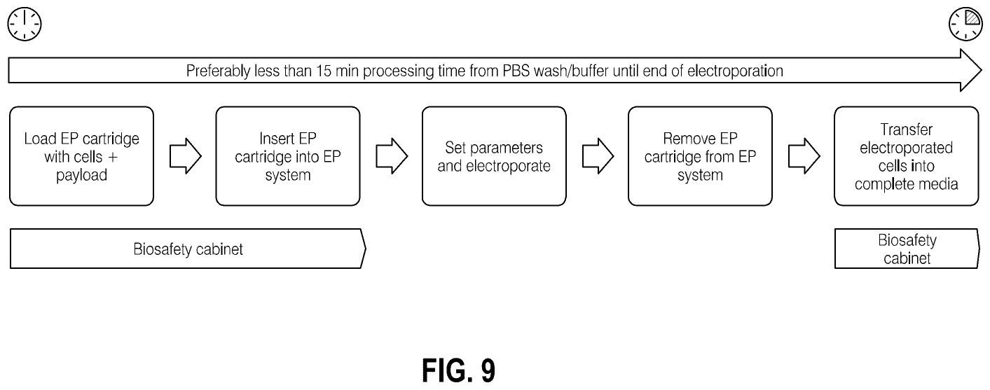

As shown in the exemplary electroporation protocol of FIG. 9, an aliquot or total volume of cells prepared for testing is loaded into an electroporation (EP) cartridge along with a desired payload or electroporation target. The EP cartridge is inserted into an electroporation system, such as those described herein configured to batch process samples, electroporation parameters are set and executed on the system, and the EP cartridge is removed from the system. The electroporated cells can then be transferred into complete media or other recovery media and incubated for a period of time (e.g., 24-72 hours). The electroporated cells can then be interrogated for viability and electroporation efficiency using an appropriate biochemical, optical, or molecular readout (e.g., Western blot for protein concentration/expression, flow cytometry for expression of transformed fluorescent protein, qPCR for molecular analysis, etc.). Other measures of electroporation level or efficiency can include various metrics, such as: efficiency of electroporation, cell viability after electroporation, expression of gene or protein of interest in electroporated cell, knockout of a gene of interest, or change in genotype or phenotype of a cell. Combinations of the foregoing are possible as well. The current disclosure is not intended to be limited to any particular measure of electroporation levels, efficiency or cellular quality or status.

User interface 3810 of electroporation system 3800 of FIG. 3 can take a variety of forms. A readable and manipulable touch screen is preferred. The user interface can comprise LCD (liquid crystal display), LED (light emitting diode), OLED (organic light emitting diode), or any appropriate screen technology. Touch manipulation is preferred. A keyboard, toggle, joystick, or other input means is possible as well.

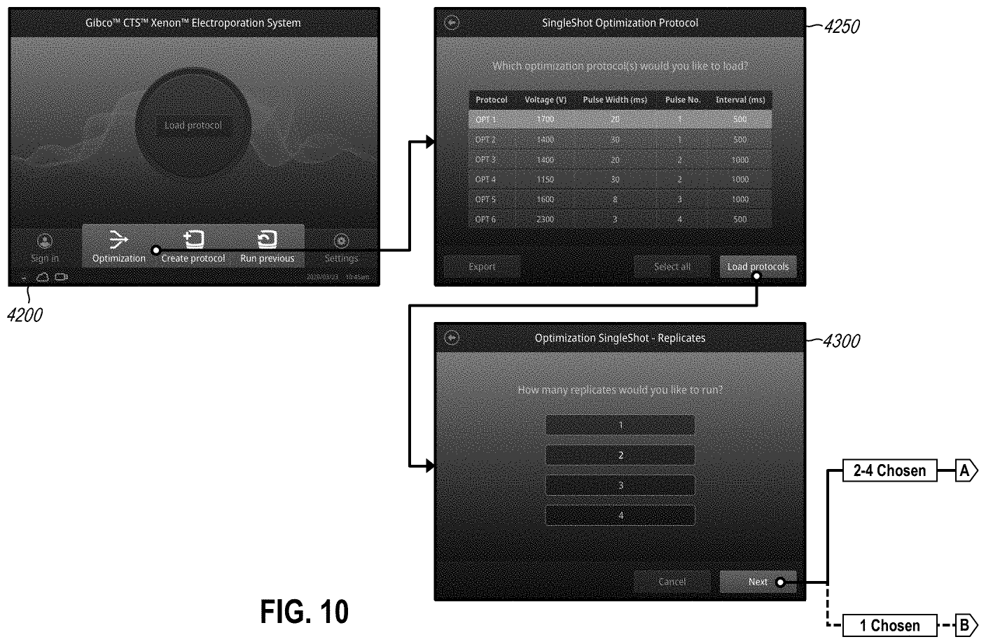

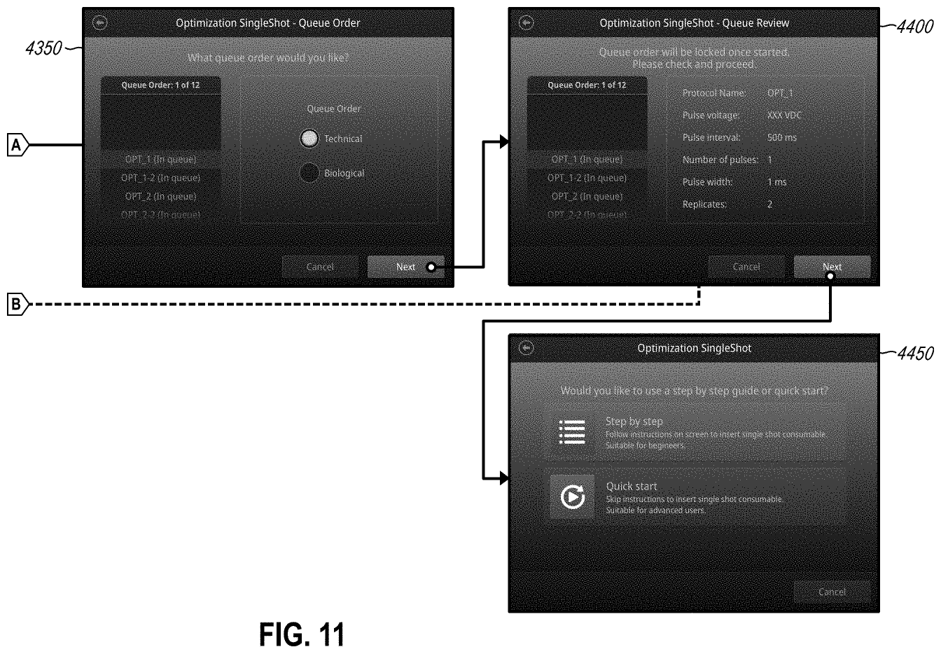

FIGS. 10 and 11 show possible user interfaces and queues allowing a user to interact with electroporation system 3800 of FIG. 3. FIGS. 10 and 11 also help to illustrate methods of using the electroporation system and possible electroporation protocol optimization parameters and uses. FIG. 10 shows a possible home page 4200, or introductory page for use of an electroporation machine. Home page 4200 can give a user a plurality of options such as sign in, optimization, create protocol, run previous, and settings. Clicking on optimization can bring a user to optimization protocol page 4250. Optimization protocol page 4250 allows the user to select which optimization protocols to run. As seen, each optimization protocol OPT 1 to OPT 6 comprises a unique combination of voltage, pulse width, pulse number, and interval. Other parameters could be included as well. From optimization protocol page 4250, the user can press “load protocol” and move to replicate page 4300. Here the user can select a number of replicates to run. This can allow for multiple trials for each optimization protocol. Results of multiple replicates can be averaged to provide more accurate results. Pressing ‘next,’ the user can proceed to queue order page 4350 of FIG. 11 and choose how to order the various trials and optimization protocols being run. Queue can be arranged by technical order, biological order, or another factor. Pressing ‘next,’ the user can proceed to queue review page 4400 to review the queue order. Pressing ‘next,’ the user can proceed to page 4450 to select between a step-by-step guidance to running trials or quick start.

Arranging the queue by technical order can refer to an order of technical replicates that can be used to access reproducibility of results across the instrument, platform, software, or other hardware or software components. For example, a single sample may be divided into multiple samples and tested to check if systems or software performs uniformly. In another example, the same cells can be tested in different types of cartridges or consumables to check if results are uniform. In another example, performance of systems or software can be assessed when users of different technical abilities/expertise use the same instrument. Arranging the queue by biological order can refer to using biological replicates to test different biological samples. For example, one cell population can be tested, but over the course of a number of days. Some cells are tested on day 1, some others on day 2, some others on day 3, and so forth. Note that some cells will have different properties since they are not identical cells. In another example, some cells can be tested before and after a treatment (such as with a chemical/heat/other condition)

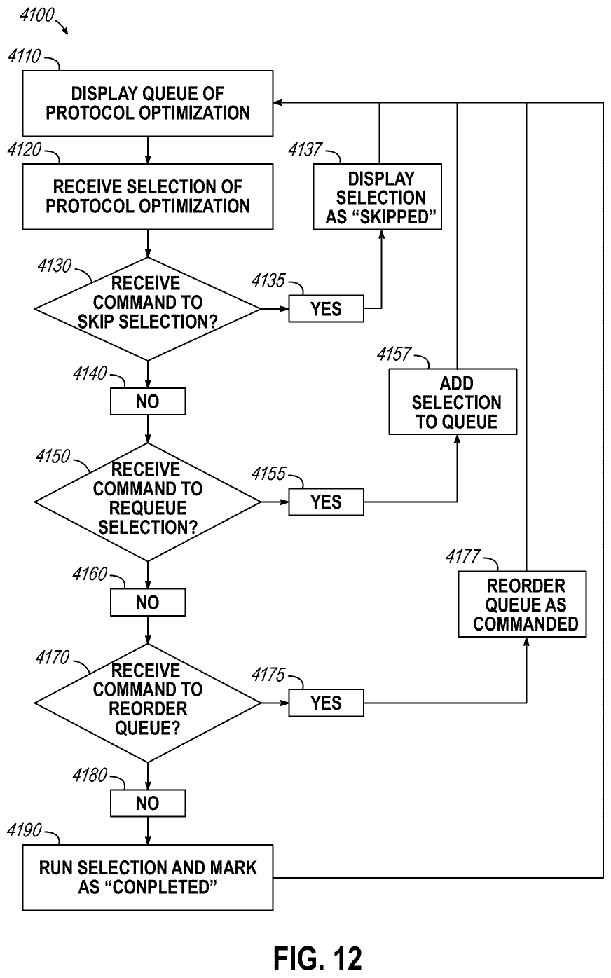

One unique aspect of the described embodiments is the ability to monitor the queue of trials or optimization protocols that are to be run. Furthermore, the ability to skip and repeat trials, to ‘requeue’ a trial, and to have such status be updated in the queue gives the user increased ability to manage the trials being run. A flow-chart illustrating a method of skipping, requeuing, and ordering the queue is shown in FIG. 12. In method 4100, at 4110 the queue of protocol optimizations is displayed to a user. At 4120, a selected protocol optimization is received. At 4130, if there's a command to skip the selection 4135, then the selection is marked as “skipped” 4137 and the process returns to 4110. If there's no skip 4140, then it is determined if there's a command to requeue the selection 4150. If yes 4155, then the selection is added to the queue, and any “skipped” label is removed 4157 and the process returns to 4110. If there's no command to requeue 4160, then it is determined if there's a command to reorder the queue 4170. If yes 4175, then the queue is reordered as commanded 4177, and then the process returns to 4110. If there's no reorder command 4180, then the selected protocol optimization is run 4190 and the process returns to 4110.

### Computer Systems of the Present Disclosure

It will be appreciated that computer systems are increasingly taking a wide variety of forms. In this description and in the claims, the terms “controller,” “computer system,” or “computing system” are defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The memory may take any form and may depend on the nature and form of the computing system. The memory can be physical system memory, which includes volatile memory, non-volatile memory, or some combination of the two. The term “memory” may also be used herein to refer to non-volatile mass storage such as physical storage media.

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof.

For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled— whether in a single stage or in multiple stages—so as to generate such binary that is directly interpretable by a processor.

The term “executable component” is also well understood by one of ordinary skill as including structures that are implemented exclusively or near-exclusively in hardware logic components, such as within a field programmable gate array (FPGA), an application specific integrated circuit (ASIC), Program-specific Standard Products (ASSPs), System-on-a-chip systems (SOCs), Complex Programmable Logic Devices (CPLDs), or any other specialized circuit. Accordingly, the term “executable component” is a term for a structure that is well understood by those of ordinary skill in the art of computing, whether implemented in software, hardware, or a combination thereof.

The terms “component,” “service,” “engine,” “module,” “control,” “generator,” or the like may also be used in this description. As used in this description and in this case, these terms whether expressed with or without a modifying clause—are also intended to be synonymous with the term “executable component” and thus also have a structure that is well understood by those of ordinary skill in the art of computing.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

Accordingly, embodiments described herein may comprise or utilize a special purpose or general-purpose computing system. Embodiments described herein also include physical and other computer-readable media for carrying or storing computer-executable instructions and/or data structures. Such computer-readable media can be any available media that can be accessed by a general purpose or special purpose computing system. Computer-readable media that store computer-executable instructions are physical storage media. Computer-readable media that carry computer-executable instructions are transmission media. Thus, by way of example—not limitation—embodiments disclosed or envisioned herein can comprise at least two distinctly different kinds of computer-readable media: storage media and transmission media.

Computer-readable storage media include RAM, ROM, EEPROM, solid state drives (“SSDs”), flash memory, phase-change memory (“PCM”), CD-ROM or other optical disk storage, magnetic disk storage or other magnetic storage devices, or any other physical and tangible storage medium that can be used to store desired program code in the form of computer-executable instructions or data structures and that can be accessed and executed by a general purpose or special purpose computing system to implement the disclosed functionality of the invention. For example, computer-executable instructions may be embodied on one or more computer-readable storage media to form a computer program product.

Transmission media can include a network and/or data links that can be used to carry desired program code in the form of computer-executable instructions or data structures and that can be accessed and executed by a general purpose or special purpose computing system. Combinations of the above should also be included within the scope of computer-readable media.

Further, upon reaching various computing system components, program code in the form of computer-executable instructions or data structures can be transferred automatically from transmission media to storage media (or vice versa). For example, computer-executable instructions or data structures received over a network or data link can be buffered in RAM within a network interface module (e.g., a “NIC”) and then eventually transferred to computing system RAM and/or to less volatile storage media at a computing system. Thus, it should be understood that storage media can be included in computing system components that also—or even primarily—utilize transmission media.

Those skilled in the art will further appreciate that a computing system may also contain communication channels that allow the computing system to communicate with other computing systems over, for example, a network. Accordingly, the methods described herein may be practiced in network computing environments with many types of computing systems and computing system configurations. The disclosed methods may also be practiced in distributed system environments where local and/or remote computing systems, which are linked through a network (either by hardwired data links, wireless data links, or by a combination of hardwired and wireless data links), both perform tasks. In a distributed system environment, the processing, memory, and/or storage capability may be distributed as well.

Those skilled in the art will also appreciate that the disclosed methods may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

### Abbreviated List of Defined Terms

To assist in understanding the scope and content of this written description and the appended claims, a select few terms are defined directly below. Unless defined otherwise, all technical and scientific terms used herein have the same meaning as commonly understood by one of ordinary skill in the art to which the present disclosure pertains.

The terms “approximately,” “about,” and “substantially,” as used herein, represent an amount or condition close to the specific stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a specifically stated amount or condition.

As used herein, the term “electroporation” is intended to include the process of exposing cells to an electric field, typically a short duration, high voltage electric field, to cause the uptake of an electroporation target from the surrounding electroporation media into the electroporated cell. The cell can be any living cell, and it should be appreciated that the electroporation systems and methods disclosed herein can be used with prokaryotic and/or eukaryotic cells. As known to those skilled in the art, the process of electroporating a target into a prokaryotic organism, such as a bacterium, is termed “transformation,” whereas electroporating a target into a eukaryotic organism, such as primary cells or cell lines, is typically termed “transfection.” For the purposes of this disclosure, the terms “transformation” and “transfection” are interchangeable and agnostic to the type or kind of organism being transformed, unless specifically stated otherwise. Accordingly, the term “electroporation,” or forms thereof, is intended to include the transformation/transfection of living cells—prokaryotic or eukaryotic—with an electroporation target.

The term “electroporation target,” as used herein, is intended to be understood as any molecule, compound, or substance intended to be introduced to a target cell via electroporation. By way of example and not limitation, an electroporation target can include, proteins, peptides, nucleic acid, drug, or another compound. Proteins can include purified, folded, or unfolded proteins having a native, mutated, or engineered sequence, and peptides are understood to include any string of amino acids and may comprise portions of a protein sequence. Nucleic acid includes those sequences derived from a biological or environmental source and can be one or more of a gene, a regulatory sequence, intergenic sequence, genomic DNA, plasmid DNA, cDNA, or any of the various known forms of RNA. As is outlined herein, the electroporation target may take any of the foregoing forms, although in a preferred embodiment, the electroporation target constitutes a nucleic acid for transfecting primary cells or cell line.

As used herein, the term “primary cell” is intended to denote cells isolated directly from the tissue or bodily fluid of an organism that, without intervention, have a finite lifespan and limited in vitro expansion capacity using standard cell culture techniques. Primary cells are typically not associated with homogenous genotypic and phenotypic characteristics. In contrast to primary cells, the term “cell line” is intended to include those cells that that have acquired homogenous genotypic and phenotypic characteristics (e.g., from continual passaging over a long period of time). As known by those having skill in the art, cell lines include finite or continuous cell lines. An immortalized or continuous cell line has acquired the ability to proliferate indefinitely, either through genetic mutations or artificial modifications.

The term “sealing member,” as used herein, is intended to include any structural element or mechanism known in the art that facilitates, forms, or acts to seal the junction between two surfaces. The sealing members disclosed herein preferably include O-rings or other gaskets that selectively allow the disclosed and associated electroporation cartridges to act as a functionally closed environment. The O-rings or similar gaskets provided within the scope of this disclosure can be made of or include any suitable material known in the art, including by way of example and not limitation, a non-conductive material, such as rubber or silicone.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used in the specification, a word appearing in the singular encompasses its plural counterpart, and a word appearing in the plural encompasses its singular counterpart, unless implicitly or explicitly understood or stated otherwise. Thus, it will be noted that, as used in this specification and the appended claims, the singular forms “a,” “an” and “the” include plural referents unless the context clearly dictates otherwise. For example, reference to a singular referent (e.g., “a widget”) includes one, two, or more referents unless implicitly or explicitly understood or stated otherwise. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

As used herein, directional terms, such as “top,” “bottom,” “left,” “right,” “up,” “down,” “upper,” “lower,” “proximal,” “distal,” “adjacent,” and the like are used herein solely to indicate relative directions and are not otherwise intended to limit the scope of the disclosure and/or claimed invention.

### CONCLUSION

It is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention itemed. Thus, it should be understood that although the present invention has been specifically disclosed in part by preferred embodiments, exemplary embodiments, and optional features, modification and variation of the concepts herein disclosed may be resorted to by those skilled in the art, and such modifications and variations are considered to be within the scope of this invention as defined by the appended items. The specific embodiments provided herein are examples of useful embodiments of the present invention and various alterations and/or modifications of the inventive features illustrated herein, and additional applications of the principles illustrated herein that would occur to one skilled in the relevant art and having possession of this disclosure, can be made to the illustrated embodiments without departing from the spirit and scope of the invention as defined by the items and are to be considered within the scope of this disclosure.

It will also be appreciated that systems, devices, products, kits, methods, and/or processes, according to certain embodiments of the present disclosure may include, incorporate, or otherwise comprise properties or features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

All references cited in this application are hereby incorporated in their entireties by reference to the extent that they are not inconsistent with the disclosure in this application. It will be apparent to one of ordinary skill in the art that methods, devices, device elements, materials, procedures, and techniques other than those specifically described herein can be applied to the practice of the invention as broadly disclosed herein without resort to undue experimentation. All art-known functional equivalents of methods, devices, device elements, materials, procedures, and techniques specifically described herein are intended to be encompassed by this invention.

When a group of materials, compositions, components, or compounds is disclosed herein, it is understood that all individual members of those groups and all subgroups thereof are disclosed separately. When a Markush group or other grouping is used herein, all individual members of the group and all combinations and sub-combinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. All changes which come within the meaning and range of equivalency of the items are to be embraced within their scope.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An electroporation system configured to test for optimal parameters for the electroporation of a sample, the electroporation system comprising:
a compartment configured to removably receive one electroporation chamber, the electroporation chamber comprising a portion of the sample, the sample comprising one or more particular cell types; and
a controller communicatively coupled to the electroporation chamber, wherein the controller is configured to run one or more optimization routines, each optimization routine including a set of parameters for the electroporation of the sample to determine the optimal parameters for electroporation of the sample;
wherein one optimization routine is run on the portion of the sample in the electroporation chamber.

2. The electroporation system of claim 1, wherein the set of parameters includes voltage, pulse width, pulse number, buffer type, pulse type, and pulse interval.

3. The electroporation system of claim 2 wherein pulse type refers to positive or negative polarity pulse.

4. The electroporation system of claim 1, wherein the controller is further configured to allow selection of a number of replicates for a selected optimization routine.

5. The electroporation system of claim 1, wherein the controller is further configured to create a queue of selected optimization routines.

6. The electroporation system of claim 1, wherein the controller is further configured to repeat individual optimization routines in the queue of selected optimization routines.

7. The electroporation system of claim 1, wherein the controller is further configured to skip individual optimization routines in the queue of selected optimization routine.

8. The electroporation system of claim 1, wherein the controller is further configured to re-order the selected optimization routines.

9. The electroporation system of claim 1, wherein the controller is configured to display a step-by-step guide to a user.

10. The electroporation system of claim 1, wherein the controller is configured to display a “quick start” option to a user, wherein the “quick start” option avoids a step-by-step guide.

11. The electroporation system of claim 1 wherein the electroporation chamber is within an electroporation cartridge, the electroporation cartridge comprising two electrodes.

12. The electroporation system of claim 11 wherein the electroporation cartridge comprises at least one resealable cap.

13. The electroporation system of claim 12 wherein at least one of the electrodes of the electroporation cartridge comprises at least part of the resealable cap.

14. The electroporation system of claim 1, wherein the electroporation cartridge comprises a volume reducing sleeve.

15. The electroporation system of claim 11 wherein the electroporation cartridge comprises at least one fluid overfill reservoir.

16. The electroporation system of claim 11 wherein the electroporation cartridge comprises an authentication chip.

17. The electroporation system of claim 15 wherein the authentication chip comprises an RFID chip.

18. The electroporation system of claim 15 wherein the authentication chip comprises any of an erasable programmable read-only memory (EPROM), an electrically erasable programmable read-only memory (EEPROM), or flash memory.

19. The electroporation system of claim 15 wherein the authentication chip comprises a non-floating-gate technology capable of surviving gamma radiation.

20. The electroporation system of claim 11 wherein the electroporation chamber comprises:
an elongate body;
a first electrode disposed at a proximal end of the electroporation chamber; and
a second electrode disposed at an opposite, distal end of the electroporation chamber.

21. The electroporation system of claim 11 wherein the electroporation chamber comprises one or more of a non-conductive plastic, a glass, and/or a ceramic and is configured to receive a cell-containing liquid to be electroporated within the electroporation chamber.

22. The electroporation system of claim 19 wherein at least a portion of the electroporation chamber is tapered between the first electrode and the second electrode.

23. The electroporation system of claim 1, wherein the controller is configured to receive a command from a user to skip an optimization protocol.

24. The electroporation system of claim 2, wherein the controller is configured to receive a command from a user to add the skipped optimization back to a queue.

25. The electroporation system of claim 1 wherein a second optimization routine is run on a second portion of the sample in a successively received electroporation chamber.

26. The electroporation system of claim 1 wherein the compartment removably receives one or more additional portions of the sample in the same or different electroporation chambers and wherein one or more optimization routines is run on each of the portions of samples, respectively.

27. A method of optimizing electroporation of samples comprising cells, the method comprising:
providing to a user, by an electroporation system, a list of a plurality of optimization protocols to run against one or more samples, wherein each of the plurality of optimization protocols comprises a unique combination of a plurality of parameters, wherein the electroporation system is configured to apply the plurality of parameters to the one or more samples;
receiving, from the user, a selection of one or more of the plurality of optimization protocols; and
applying, by the electroporation system in a series of operations, the one or more optimization protocols against a corresponding sample of the one or more samples such that each corresponding sample is subject to one of the one or more of the plurality of optimization protocols.

28. The method of claim 27 wherein the plurality of parameters includes voltage, pulse width, pulse number, buffer type, pulse type, and pulse interval.

29. The method of claim 27 wherein each of the one or more samples is comprised in an electroporation chamber.

30. The method of claim 27 wherein each of the one or more samples comprises an authentication chip or is part of an electroporation cartridge that comprises an authentication chip.

31. The method of claim 30 wherein the authentication chip comprises an RFID chip.

32. The method of claim 30 wherein the authentication chip comprises any of an erasable programmable read-only memory (EPROM), an electrically erasable programmable read-only memory (EEPROM), or flash memory.

33. The method of claim 30 wherein the authentication chip comprises a non-floating-gate technology capable of surviving gamma radiation.

34. The method of claim 27 further comprising receiving, by the electroporation system, a number of replicates to run and applying, for each optimization protocol, the number of replicates against one of the one or more samples.

35. The method of claim 27 further comprising displaying, to a user, instructions on opening the system, inserting an electroporation cartridge into the system, closing the system and running the optimization protocols.

36. The method of claim 27 further comprising creating a queue of selected optimization routines.

37. The method of claim 36 further comprising receiving a request, from a user, to repeat a selected optimization routine in the queue.

38. The method of claim 36 further comprising receiving a request, from a user, to skip a selected optimization routine in the queue.

39. The method of claim 36 further comprising receiving a request, from a user, to re-order the selected optimization routines in the queue.

40. The method of claim 36 further comprising displaying, to a user, a status of each selected optimization routine in the queue.

41. The method of claim 40 wherein status can be at least one of; completed, skipped, and in progress.

42. The method of claim 27 wherein the one or more samples comprise one or more portions of a sample.

43. A method for optimizing electroporation of a sample comprising cells, the method comprising:
receiving, by an electroporation system, a choice of one or more optimization protocols to run against one or more samples, wherein each of the one or more optimization protocols comprises at least a voltage, a pulse width, and a number of pulses;
receiving, by the electroporation system, a number of replicates to run, wherein the number of replicates multiplied by the one or more optimization protocols equals a number of trials;
receiving, by the electroporation system in a series of electroporation operations corresponding to a number of selected optimization protocols and the number of replicates, an electroporation chamber containing one of the one or more samples for each of the number of trials; and
applying to each respective sample, by the electroporation system, the respective optimization protocol.

44. The method of claim 43 wherein each of the one or more samples is received in an electroporation cartridge.

45. The method of claim 43 further comprising sending, by the electroporation system, results of each of the one or more samples to a remote machine for analysis.

46. The method of claim 43 wherein each of the one or more samples is contained in an electroporation chamber of an electroporation cartridge.

47. The method of claim 43 wherein each of the one or more samples comprises a cell population having a particular cell type.

48. The method of claim 43 wherein each of the one or more samples comprises a cell population having a plurality of cell types.

49. The method of claim 43 further comprising:
assessing, after each trial, a level of electroporation in cells of the respective one or more samples; and
assessing, after all trials have completed, which optimization protocol achieved the best electroporation.

50. The method of claim 43 further comprising assessing, after each trial, one or more of electroporation efficiency, cell viability, and expression of electroporated cells.

51. The method of claim 43 wherein the electroporation system is configured to assess risk of arcing for each optimization protocol.

52. The method of claim 43 further comprising recording, by the electroporation system, information from an authentication tag associated with each of the one or more samples.

53. The method of claim 43 wherein the one or more samples comprise one or more portions of a sample.
